(19) United States
(12) Plant Patent Application Publication
(10) Pub. No.: US 2022/0201911 P1
(43) Pub. Date: Jun. 23, 2022

(54) MANDARIN TREE NAMED '12C009'

(50) ;

(71) Applicant: State of Queensland Through the Department of Agriculture and Fisheries, Brisbane (AU)

(72) Inventor: Malcolm W. SMITH, Bundaberg (AU)

(73) Assignee: State of Queensland Through the Department of Agriculture and Fisheries, Brisbane (AU)

(21) Appl. No.: 17/546,834

(22) Filed: Dec. 9, 2021

Related U.S. Application Data
(60) Provisional application No. 63/128,532, filed on Dec. 21, 2020.

(51) Int. Cl. A01H 6/78

(52) U.S. Cl. PLT/202

(57) ABSTRACT

A new and distinct variety of mandarin tree named ‘12C009’, particularly selected for non-acidic flavour profiles and distinguished by the smooth skin texture, cleanness of peeling and high Brix levels, is disclosed.

### CROSS-REFERENCE TO RELATED APPLICATION

This application claims the benefit of U.S. Provisional Application No. 63/128,532, filed Dec. 21, 2020, which is incorporated herein by reference in its entirety.

Latin name: Botanical classification: Citrus reticulata.

Varietal denomination: The varietal denomination of the claimed variety of mandarin tree is ‘12C009’.

### BACKGROUND OF THE INVENTION

Mandarin is an important and valuable commercial fruit crop. Accordingly, there is a need for new varieties of mandarin trees. In particular, there is a need for improved varieties that can be harvested late in the season and meet consumer expectations for attractive external appearance (e.g. color and smoothness) combined with good eating qualities (e.g. sweet and low-seeded).

### SUMMARY OF THE INVENTION

In order to meet these needs, the present invention is directed to an improved variety of mandarin tree. In particular, the invention relates to a new and distinct variety of mandarin tree (Citrus reticulata), which has been denominated as ‘12C009’.

The initial step in the invention involved selection from a breeding population of approximately 6,000 hybrids between ‘Ellendale’ (unpatented) and ‘Murcott’ (unpatented), which was made in 2002. Following this, the material was subject to mutation breeding using a cobalt 60 gamma cell on two occasions during 2008. A small number of buds survived this treatment and developed into nursery trees suitable for field planting. A total of 147 such nursery trees (many of them double-budded) were planted at four different field sites between late 2009 and early 2011. Fruit on individual branches were checked for seed number as these trees started to come into production. ‘12C009’ was an individual branch that was identified and selected in January 2009, because it had low seed numbers good productivity and excellent quality. It was asexually propagated onto ‘Troyer’ (unpatented) rootstock and 141 resulting trees were planted at four different sites in 2016. Observation and testing of the 141 trees planted at these four sites in central Queensland, together with annual assessment (12 years) of the original limb identified in 2009, confirmed the stable retention of distinct characteristics of ‘12C009’.

‘12C009’ trees produce heavy crops of medium to large fruit that mature toward the end of the mandarin harvest season (end of July in Queensland, Australia). The fruit are firm and yet still relatively easy to peel, and the skin texture is smooth and without a prominent neck at the stem end. The fruit peel cleanly and have low seed numbers. High Brix levels are balanced with moderate acidity giving a sweet flavor well suited to Asian markets that prefer non-acidic fruit. Simulation experiments indicate that this invention is suitable for long-distance shipping while retaining its distinct mandarin characteristics.

‘12C009’ was particularly selected for its later maturity time.

### BRIEF DESCRIPTION OF THE DRAWINGS

Mandarin tree variety ‘12C009’ is shown in the accompanying photographs, illustrating typical fruit external and internal appearance, uniformity and tree productivity. The illustrated trees and fruit were grown under subtropical conditions in central Queensland, Australia and colors are as true as can be captured photographically. Field photographs are of plants that are four years old.

FIG. 1 shows fruit cross-section of ‘12C009’ (right panel) compared to the progenitor with higher seed numbers (left panel) used during the two rounds of mutation breeding.

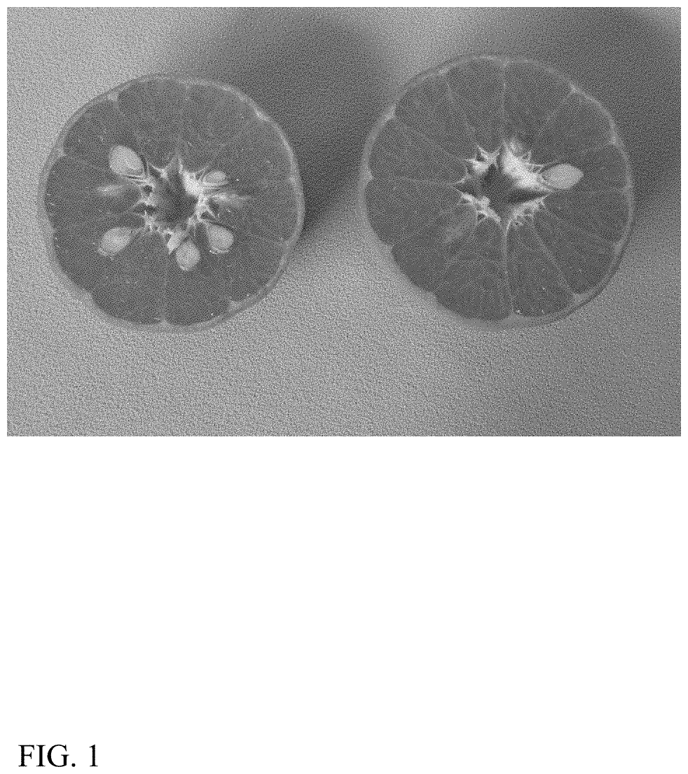

FIG. 2 shows fruit of ‘12C009’ from four different angles.

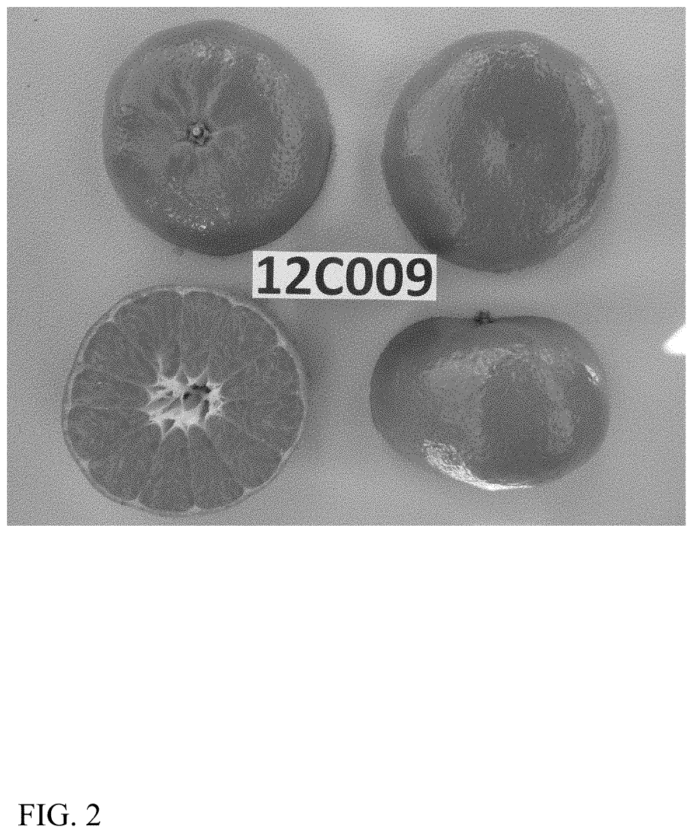

FIG. 3 shows whole fruit on a tree of ‘12C009’ along with typical leaf morphology.

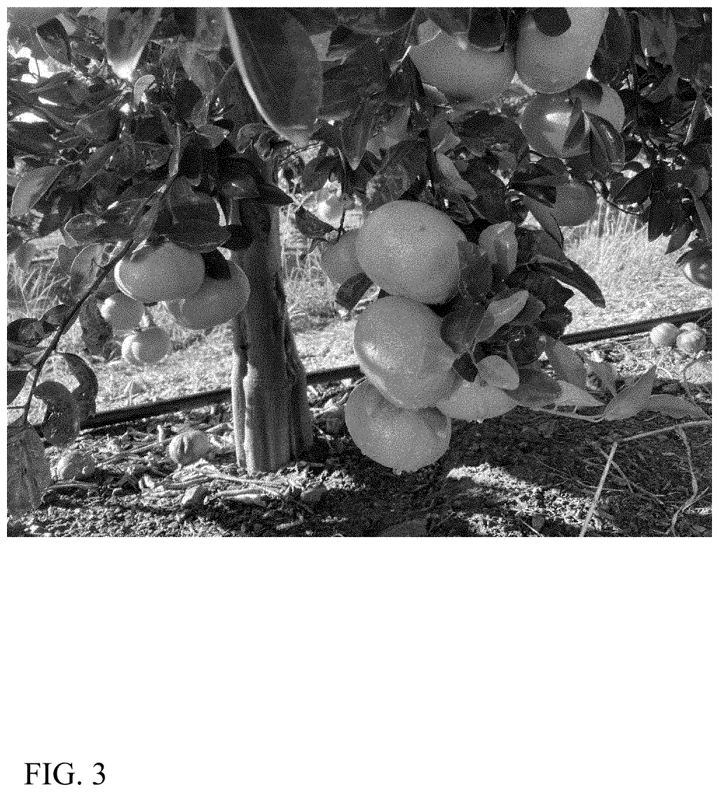

### DETAILED BOTANICAL DESCRIPTION

The following descriptions set forth the distinctive characteristics of ‘12C009’. Unless where otherwise noted, the data that define these characteristics are based on observations taken from ‘12C009’ plants that were four years old, grown on ‘Troyer’ (unpatented) rootstock in a subtropical environment at Mundubbera, Australia. Color designations, color descriptions, and other phenotypical descriptions may deviate from the stated values and descriptions depending upon variation in environmental, seasonal, climatic and cultural conditions. ‘12C009’ has not been observed under all possible environmental conditions. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.
Classification:
   Family.—Rutaceae.
   Botanical.—Citrus reticulata.
   Common name.—Mandarin.
   Variety name.—‘12C009’.
Tree:
   Ploidy.—Diploid.
   Size.—Trees have a medium to large stature.
   Vigor.—Trees grow vigorously when young but early and heavy cropping helps to restrain subsequent vigor.
   Growth habit.—Elliptic to oblong.
   Density.—Canopy is light to medium in density resulting from long branches with sometimes low crotch angles.
   Bearing.—Productive from an early age with no evidence of alternate beating.
Trunk:
   Trunk diameter.—21.5 cm at 30 cm above graft.
   Trunk texture.—Finely raised.
Branches:
   Crotch angle.—Major branches at 45-50-degree crotch angle.
   Branch length.—1.3 to 1.7 m.
   Branch texture.—Smooth becoming finely raised on older branches.
   Thorns.—Occasional on some branches.
Leaves:
   Size (lamina average).—Length: 65 mm. Width: 39 mm. L/W ratio: 1.7.
   Arrangement.—Alternate.
   Type.—Simple.
   Shape.—Obovate.
   Blade margin.—Crenate.
   Apex shape.—Obtuse, marginated at tip.
   Petiole length.—10 mm.
   Petiole diameter.—2 mm.
   Petiole wings.—Rudimentary <1 mm width.
   Petiole attachment.—Articulate, brevipetiolate.
Flowers:
   Type.—Hermaphroditic.
   Blooming period.—Commencing˜25th August with full bloom˜24th September in Queensland, Australia.
   Flower petals.—Number: 5. Color: RHS 155B (white). Length: 11 mm. Width: 4 mm. Calyx: Small.
   Fragrance.—Typical of mandarin.
   Reproductive organs.—Fertility: Self-fertile. Pollen amount: Moderately abundant. Pollen fertility: Low (assessed via aceto-carmine staining).
Fruit:
   Axial diameter.—87 mm.
   Apical diameter.—53 mm.
   Weight.—196 g.
   General shape in profile.—Obloid.
   Shape in transverse section.—Somewhat angular.
   Shape of fruit base.—Truncate, occasionally with wrinkles.
   Shape of fruit apex.—Truncate.
   Neck.—Absent.
   Fruit surface texture.—Smooth.
   Adherence of mesocarp to endocarp.—Weak and peels away cleanly.
   Navel.—Absent.
   Areola.—Generally absent but occasionally grooved.
   Rind thickness.—3.3 mm.
   Segment number.—10-12.
   Harvest window.—Late-season: end-July (southern hemisphere subtropics).
   Juice soluble solid.—12.4°Brix (end-July).
   Juice acidity.—0.95% citric acid equivalent (end-July).
   Seeds.—0-5 per fruit.
   Seed type.—Polyembryonic.
   Alternaria brown spot reaction.—Susceptible.

What is claimed is:

1. A new and distinct mandarin tree called ‘12C009’ as illustrated and described herein.
